(19) United States
(12) Patent Application Publication
(10) Pub. No.: US 2025/0312291 A1
(43) Pub. Date: Oct. 9, 2025

(54) DIARYLHEPTANOID BASED MEDICINAL PREPARATION AND METHOD OF PREPARATION OF THE SAME

(71) Applicant: ARJUNA NATURAL PRIVATE LIMITED, Kerala (IN)

(72) Inventor: Benny ANTONY, Kerala (IN)

(21) Appl. No.: 18/865,201

(22) PCT Filed: May 12, 2023
(86) PCT No.: PCT/IB2023/054917

§ 371 Date: Nov. 12, 2024

(30) Foreign Application Priority Data
May 13, 2022 (IN) 202241027627

(51) Int. Cl. A61K 31/12; A61K 9/16; A61K 33/06; A61K 33/30; A61K 36/9066; A61P 1/16; A61P 3/00; A61P 3/14; A61P 29/00; A61P 33/10; A61P 37/04; A61P 43/00

(52) U.S. Cl. CPC A61K 31/12; A61K 9/1682; A61K 33/06; A61K 33/30; A61K 36/9066; A61P 1/16; A61P 3/00; A61P 3/14; A61P 29/00; A61P 33/10; A61P 37/04; A61P 43/00; A61K 2236/35; A61K 2236/51; A61K 2236/53

(57) ABSTRACT

The disclosure pertains to a medicinal preparation having a mixture of diarylheptanoid complexes and essential oil of turmeric embedded in diarylheptanoid metal complex. It also discloses a method of preparation in which the complex is formed by the reaction of diarylheptanoids with a cation in the presence of oil. Diarylheptanoid metal complexes with embedded oil show improvement in the general health of avians and mammals. Improvements in avians and mammals health include reduced mortality, improved weight gain, improved meat quality, improved bone density, improved eggshell thickness, improved immunomodulatory activity, improved liver conditions, and reduced occurrence of drug-induced dyslipidemia. It is also found that said medicinal preparation is very effective in the prevention of the incidence of udder inflammation and in the treatment of hypocalcemia, Alzheimer's disease, and stress related issues. It has antioxidant activity as well as improved analgesic properties.

### FIELD OF THE INVENTION

The present invention relates to medicinal preparation comprising a free-flowing stain free composition having a mixture of diarylheptanoid complexes and essential oil of turmeric wherein said oil is embedded in diarylheptanoid metal complex. It also discloses a method of preparation and application of said medicinal preparation containing organic active ingredients forming salts or complexes with metals and minerals.

### BACKGROUND OF THE INVENTION

Diarylheptanoids are a relatively small class of plant secondary metabolites with a wide variety of bioactivity with low toxicity. One of the well-known diarylheptanoids is curcumin, which is obtained from the rhizome of the plant Curcuma longa (turmeric). Curcumin from turmeric is used for its medicinal activity and colouring ability. Curcumin is the major metabolite found in turmeric rhizome and has proven medical property. Studies have shown that other metabolites of turmeric such as bisdemethoxycurcumin and demethoxycurcumin also have significant bioactivity.

Curcumin is also used in the food industry for its colouring property. Reference is made to U.S. Pat. No. 4,263,333 which discloses curcumin-stannous and curcumin-zinc complex as colouring pigments. The colouring property of curcumin pigment makes it difficult to use in industry, as curcuminoids have poor bioavailability. There are few attempts to make metal conjugates of curcumin, U.S. Pat. No. 8,759,562 is directed towards a curcumin-Zinc complex for treatment of dementia. U.S. Pat. No. 4,263,333 is directed towards a curcumin-stannous and curcumin-zinc complex to be used as colouring pigments. US patent application 2009/0098226 is directed towards a cosmetic formulation with curcumin and metal salts, where there can be a curcumin-metal complex in the formulation. The prior art does not teach about medicinal use of metal complex, especially in the poultry industry or for animal health. Further, most of the teaching from the prior arts are limited to curcumin only and other diarylheptanoids are mostly ignored.

Few documents pertain to diarylheptanoids other than curcumin, such as European patent 0839037, which is directed towards a formulation containing curcuminoids and curcuminoid complex for antimicrobial activity. European patent 2694020 is directed towards an oral composition in which curcumin and its analogy complex are used for dental health. But the free-flowing stain free composition having a mixture of diarylheptanoid complexes and oil wherein said oil is embedded in diarylheptanoid metal complex is not known in the art. Further, the benefit of oil on metal-complex is also not known earlier.

There is a need for a diarylheptanoid a metal complex having embedded oil which has medicinal use and a commercially viable method to manufacturing the same.

### OBJECTS OF THE INVENTION

The principal object of this invention is to manufacture a free-flowing diarylheptanoids complex with embedded oil in it.

Another objective of the invention is the medicinal use of such a diarylheptanoids complex.

### SUMMARY

The invention relates to a medicinal preparation comprising a free-flowing stain free composition having a mixture of diarylheptanoid complex and essential oil of turmeric embedded in diarylheptanoid metal complex wherein the diarylheptanoid complex is having the Formula.

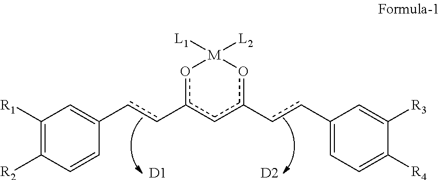

Wherein, R₁, R₂, R₃, and R₄ are selected from a group consisting of: hydrogen, —OH, —OCH₃, —OCOCH₃, —OCO—CHR—NH₂, O-Piperoyl, —O-galloyl and —O-terpenoid.

L1 and L2 are selected from a group consisting of —OH, —OH₂, CI, amine, Diarylheptanoids, flavonoids, phenolic acids, stilbenes, saponins and tannins;
D1 and D2 are selected from a group consisting of single and double bonds; and
M is selected from a group consisting of alkali metals, alkaline earth metal, transition metals, lanthanides, and actinides.

Said composition is having at least 5% essential oil of turmeric by weight and preferably between 5 to 40% and more preferably 20-25% in embedded form in the diarylheptanoid complex. It is found that diarylheptanoid metal complex makes 1% to 90% of said composition and preferably between 60% to 90% of said composition.

The medicinal preparation comprising a free-flowing stain free composition having a mixture of diarylheptanoid complex and essential oil of turmeric embedded in diarylheptanoid metal complex. According to our invention, the general health of avians and mammals is found to improve by administering the said medical preparation. The general health of avians and mammals includes but is not limited to reduced mortality, improved weight gain, improvement in meat quality, preventing hypocalcaemia, improvement in bone density, improved eggshell thickness, improved immunomodulatory activity, improved hepatoprotective activity, improved the analgesic property, preventing mastitis in milch animals, preventing drug-induced dyslipidacmia, treating stress, treating Alzheimer's disease, antioxidant activity. It is found that said diarylheptanoid complex showed significant results in treating coccidia infection. It can also be administered to control the worms in mammals and avians. It can also be administered to improve the liver conditions and it works as an immunomodulator. The medicinal preparation is useful for reducing the mortality, improving the weight gain, preventing hypocalcaemia in mammals and in avians. The medicinal preparation is also effective in improving the shell quality of eggs. The medicinal preparation prevents the incidence of udder inflammation in mammals.

Said medicinal preparation can be administered alone, or it can be mixed with a pharmaceutically-acceptable carrier or diluent depending on the mode of administration to avians and mammals. It can be administered orally, parenterally and topically. It can be administered orally in the form of tablets, capsules, granules, powders, lozenges, syrups, elixirs, solutions, and suspensions following the standard pharmaceutical practice. Carriers useful in formulating the preparations are commonly used pharmaceutically acceptable non-toxic carriers such as gelatine, lactose, sodium citrate, salts of phosphoric acid, starch, magnesium stearate, sodium lauryl sulphate, talc, and polyethene glycol. Said carrier may be used with other additives such as diluents, binders, buffer agents, preservatives, sweetening agents, flavouring agents, glazes, disintegrators, coating agents, emulsifying agents, and suspending agents. Parenteral administration includes intramuscular, intraperitoneal, subcutaneous and intravenous use. Effective dosage of said medicinal preparation is between 50-1000 mg two times a day.

We also disclose a method of preparing said medicinal preparation having a mixture of diarylheptanoid complex with essential oil of turmeric in embedded form in diarylheptanoid metal complex having the Formula-1 comprising of the following steps:
a) extracting the cleaned Turmeric rhizome with an organic solvent, preferably with ethyl acetate to obtain a solvent part and residue part;
b) separating the solvent part from the residue, and concentrating to obtain the oleoresin;
c) cooling said oleoresin to a temperature of 4° C. to crystalize curcuminoids separating the curcuminoid crystals from the oleoresin;
d) dissolving the remaining oleoresin in a methanol to get a solution;
e) adding a source of calcium dispersed in water to the resultant solution of step d);
f) maintaining the resultant solution at a temperature of 27° C. and stirring till precipitate is formed; and
g) separating the precipitate, washed with water and methanol and drying to obtain a diarylheptanoid complex of calcium with embedded essential oil in the agglomerates. Here source of Calcium can be selected from lime, calcium salts and calcium oxide made from eggshell. The organic solvent used in the extraction of Turmeric rhizome is ethyl acetate while the solvent used for dissolving the remaining oleoresin in step d) is methanol.

### BRIEF DESCRIPTION OF THE DRAWINGS

Various features and advantages of the present invention should become apparent from the following figures of the accompanying drawings, which demonstrate the process steps involved in various process trials carried out under certain preferred embodiment under the invention as well as the findings.

FIG. 1 is the SEM micrograph of the diarylheptanoid complex showing the agglomerated particles and the presence voids and plate like structures.

FIG. 2 is the image of fluorescent spectrum of diarylheptanoid complex and the volatile oil is intensely coloured by fluorescent dye Nile red.

### DETAILED DESCRIPTION OF THE INVENTION

The disclosure pertains to a medicinal preparation comprising a free-flowing stain free composition having a mixture of diarylheptanoid complex and essential oil of turmeric wherein said oil is embedded in diarylheptanoid metal complex.

An advantage of the diarylheptanoid in complex form is that it is significantly easier to handle since it is stain-free. The complex is formed by the reaction of diarylheptanoids with a cation in the presence of oil. The invention also relates to the different medicinal uses of said composition.

The diarylheptanoid complex as disclosed here is in form of Formula-I as shown below.

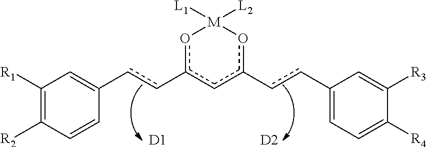

Wherein, R1, R2, R3, R4 could be any one of —OH, —OCH3, —OCOCH3, —OCO—CHR—NH2, O-Piperoyl,-O-galloyl and —O-terpenoid. L1 and L2 could be any one of OH, —OH2, Cl−, amine, flavonoids, phenolic acids, stilbenes, saponins and tannins or a similar Diarylheptanoids. D1 and D2 are either single or double bonds, and M is the cation. The cation is selected from alkali metals, alkaline earth metals, transition metals, lanthanides, and actinides. More specifically a suitable metal cation forming a bond with the diketone of diarylheptanoid is shown in Formula-1. Examples of such metal cations include potassium, zinc, calcium, magnesium, selenium, copper, chromium, vanadium, iron, manganese, palladium, gallium, indium, silver, tin and aluminium cations. In most case, stable structures with 1:1, 2:1 (ligand:metal) stoichiometry are observed for divalent cations.

Metal complexes are more biologically potent. Metals interact with the diarylheptanoid ligand, which brings a modification to diarylheptanoid's overall structure and improves the biological efficacy. Further, metals are known as enzyme coactivators, diarylheptanoid-metal complexes can interact with active sites of enzymes and induce multiple cellular processes. Metal complexes are more stable than diarylheptanoid in photochemical, thermal and hydrolytic degradations. Diarylheptanoid is a ligand as shown in Formula-1, and thus it forms stable metal complexes with cations inmonodendate (1:1) and bidendate (2:1) ligand to cation stoichiometry.

In the composition, the diarylheptanoid complex tends to agglomerate around oil with controlled morphological structures. When the diarylheptanoid complex is formed in presence of oil, oil is embedded during agglomeration.

The words lipids and oil may be used interchangeably throughout the specification. Oil includes essential oils from plants, oleoresins from plants, mono, sesqui, di and tri terpenoids, animal fat, and mono, di and triglycerides. More specifically, turmeric essential oil, ginger essential oil, pepper oil, Boswellia oil, sesame oil, fish oil, coconut oil, palm oil, olive oil, sunflower oil, mustard oil, lemongrass oil, bran oil, groundnut oil, lavender oil, flaxseed oil, grape seed oil, almond oil, avocado oil, soyabean oil, walnut oil, cashew oil, canola oil, corn oil, safflower oil, cottonseed oil, ramtil oil, niger oil, hemp oil, ginger oil, animal fat, medium-chain mono, di, and triglycerides or combination thereof.

In an embodiment, a composition is disclosed, the composition comprises a mixture of diarylheptanoid complexes and oil. A metal cation forms a chelate at the beta diketone moiety of the diarylheptanoid. Preferably the composition is in powdered form with oil embedded during micro agglomeration to form an oil embedded system. The diarylheptanoid complexes are as shown in Formula-I, and most of the diarylheptanoid complexes are in 1:1 and 2:1 (ligand:cation) configurations.

Further, the composition has at least 5% oil and the oil particles are between the vacant spaces of micro agglomerates of diarylheptanoid complex, because of which the composition is free-flowing even when the oil content of the composition is over 20% by weight; it can go up to 40% by weight. This is significant since if 2-part oil is added to a 3-part diarylheptanoid complex, the resulting combination is oily and will coagulate but will not form a free-flowing powder. 20-25% turmeric oil is embedded into the diarylheptanoid complex during agglomeration as evident from SEM instrumental Analysis.

### 1. SEM Analysis—

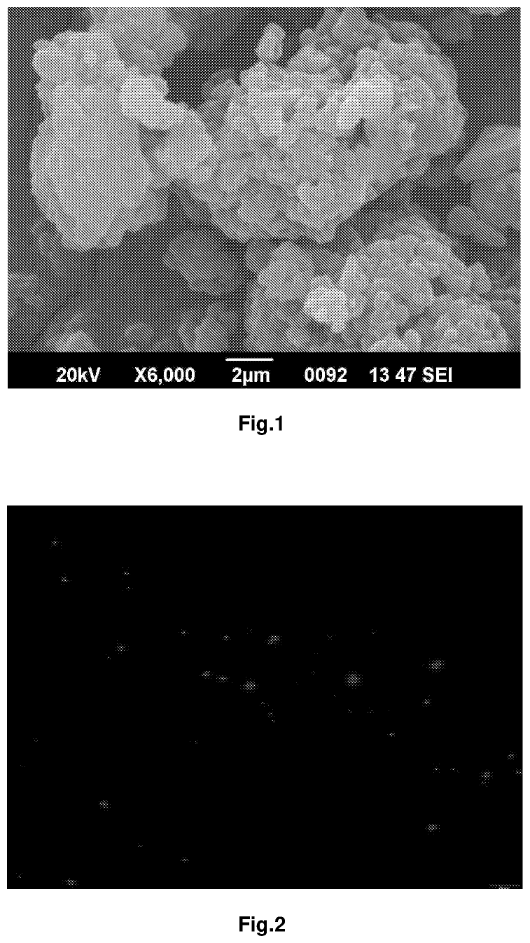

SEM micrograph of the complex (FIG. 1) shows that the particles are agglomerated and the presence voids and plate like structures. When diarylheptanoid complex is formed in presence of volatile oil, oil is embedded during agglomeration.

### 2. Florescent Spectroscopy

FIG. 2 shows the fluorescent spectrum of diarylheptanoid complex. Volatile oil embedded technology is supported by fluorescence imaging. The volatile oil is intensely coloured by fluorescent dye Nile red. Oil droplets are embedded in diarylheptanoid complex.

Further, the diarylheptanoid complexes comprise at least a curcuminoids complex, the curcuminoids complex is made of curcumin, bisdemethoxycurcumin and demethoxycurcumin complex. The curcuminoids complex makes 3% to 99% of the diarylheptanoid complexes, or curcuminoids complex makes 1% to 90% of the composition which is a mixture of diarylheptanoid complexes and oil. In one embodiment, the curcumin complex makes at most one-third of the curcuminoid complex. In yet another embodiment, the curcumin complex makes up 60% to 90% of the composition.

In an embodiment, a micro agglomeration of diarylheptanoid complexes with embedded oil in the agglomerate is disclosed. The diarylheptanoid complex as disclosed here is in form of Formula-I. wherein, R1, R2, R3, R4 could be any one of —OH, —OCH3, —OCOCH3, —OCO—CHR—NH2, O-Piperoyl, —O-galloyl and —O-terpenoid. L1 and L2 could be any one of OH, —OH2, Cl-, amine, flavonoids, phenolic acids, stilbenes, and tannins or a similar Diarylheptanoids. D1 and D2 are either single or double bonds, and M is the cation. The cation is selected from alkali metals, alkaline earth metals, transition metals, lanthanides, and actinides. More specifically a suitable metal cation forming a bond with the diketone of diarylheptanoid is shown in Formula-1. Examples of such metal cations include potassium, zinc, calcium, magnesium, selenium, copper, chromium, vanadium, iron, manganese, palladium, gallium, indium, silver, tin and aluminium cations. In general, stable structures with 1:1, 2:1 (ligand:metal) stoichiometry are formed for divalent cations.

In yet another embodiment, the composition comprises diarylheptanoid complexes of calcium and oil. The oil makes up 1 to 40% of the composition, more preferably 5 to 20% by weight. Preferably the composition is in powdered form with oil embedded between interstitial spaces of the micro agglomeration of diarylheptanoid calcium complexes. In the composition, complexes have a calcium chelating with one diarylheptanoid 1:1 (ligand:calcium) and with two diarylheptanoids in 2:1 (ligand:calcium). The oil in the composition is preferably essential oil or vegetable oil more preferably an essential oil of turmeric. In some embodiment, there can be free diarylheptanoid also present in the composition, and the free diarylheptanoid is part of the micro agglomeration. The composition has at least 1% curcuminoids calcium complexes and it can go up to 90% by weight. The curcumin complex in the curcuminoids complex can go from one third to three fourth of the curcuminoids complex.

In yet another embodiment, a composition comprises a mixture of different diarylheptanoid complexes and different oils. The mixture is of diarylheptanoid complexes with different cations. The cation can be selected from a group of potassium, zinc, calcium, magnesium, selenium, copper, chromium, vanadium, iron, manganese, palladium, gallium, indium, silver, tin and aluminium. Preferably the composition is in powdered form with oil embedded between interstitial spaces of the micro agglomeration of diarylheptanoid calcium complexes. The oil used for making each cation complex can be the same or different or even different for different cations. The oil in the composition could be an essential oil or a vegetable oil more preferably an essential oil of turmeric or ginger. In some embodiment, there can be free diarylheptanoid also present in the composition, and the free diarylheptanoid is part of the micro agglomeration. The composition has at least 1% curcuminoids calcium complexes and it can go up to 90% by weight. The curcumin complex in the curcuminoids complex can go from one third to three fourth of the curcuminoids complex. The oil used for making each cation complex can be the same or different or even different for different cations. The oil in the composition could be an essential oil or a vegetable oil more preferably an essential oil of turmeric.

The composition of the present invention can be administered alone, or it can be mixed with a pharmaceutically-acceptable carrier or diluent depending on the mode of administration. The composition of the present invention can be administered to avians and mammals. Oral administration is preferred, but parenteral and topical administration can be used. For oral administration, the composition of this invention can be used in the form of tablets, capsules, granules, powders, lozenges, syrups, elixirs, solutions, suspensions and the like, following the standard pharmaceutical practice.

The avians include but not limited to ostriches, chickens, ducks, turkeys, quail, flamingos, grebes, pigeons, mesites, sandgrouse, bustards, cuckoos, turacos, rails, cranes, waders, hoatzin, swifts, hummingbirds, nightjars, tropicbirds, kagu, loons, albatrosses, penguins, storks, cormorants, pelicans, herons, vultures, hawks, owls, quetzals, hornbills, kingfishers, woodpeckers, seriemas, falcons, parrots, and passerines.

Mammals include but not limited to rats, cats, dogs, bats, whales, dolphins, sheep, horse, camel, goat, cows, pigs and humans.

Carriers useful in formulating the preparations are commonly used pharmaceutically acceptable non-toxic carriers such as gelatine, lactose, sodium citrate, salts of phosphoric acid, starch, magnesium stearate, sodium lauryl sulphate, talc, polyethene glycol etc. The carrier may be used with other additives such as diluents, binders, buffer agents, preservatives, sweetening agents, flavouring agents, glazes, disintegrators, coating agents, emulsifying agents, suspending agents, etc.

For parenteral administration, which includes intramuscular, intraperitoneal, subcutaneous and intravenous use, sterile solutions of the active ingredients are usually prepared, and the pH of the solutions are suitably adjusted and buffered. For intravenous use, the total concentration of solutes should be controlled to render the preparation isotonic.

The daily dose of the preparation can be appropriately determined and is not particularly limited. However, in most instances, an effective dosage for an adult will be between 50-1000 mg two times a day.

Another aspect of the discloser is the method of preparing the composition. A composition containing diarylheptanoid and oil are dissolved in a solvent to obtain the first solution. Cation salt dispersed in water is slowly added to the first solution to obtain a master solution. The master solution is stirred for a fixed time at a fixed temperature. Precipitate starts forming in the master solution, the precipitate is removed and dried to get the desired diarylheptanoid complex.

In an embodiment, an economical method of producing oil embedded diarylheptanoid complex is disclosed. An oleoresin is obtained from solvent extraction of diarylheptanoid rich plant parts. The oleoresin is dissolved in a solvent, and cation salt dispersed in water is slowly added to the solution. The solution is stirred for a fixed time, at a fixed temperature. Precipitate starts forming in the solution, the precipitate is removed and dried to get the desired diarylheptanoid complex.

In an embodiment, an economical method of producing diarylheptanoid complex from turmeric is disclosed. Turmeric rhizome is extracted with an organic solvent, preferably with ethyl acetate. After extracting a solvent part and residue is obtained, the solvent part is separated from the residue, and concentrated. Lime is dispersed in water and is added to the concentrate solvent part. The solution is kept above 40° C. and stirred till precipitate is formed. The precipitate is removed and dried to obtain a diarylheptanoid complex of calcium with embedded essential oil in the agglomerates. The composition is rich in curcuminoids complex of calcium. Calcium salts could also be used instead of lime for the complex formation, a person skilled in the art will be capable of using other salts. In one embodiment, the eggshell and lime stone are used as a source of calcium and other cations.

In an embodiment, an economical method of producing diarylheptanoid complex from turmeric is disclosed. Turmeric rhizome is extracted with an organic solvent, preferably with ethyl acetate. After extracting a solvent part and residue is obtained, the solvent part is separated from the residue, and concentrated to obtain the oleoresin. The oleoresin is cooled to crystalized curcuminoids. The curcuminoid crystals are removed from the oleoresin and the remaining oleoresin is dissolved in a solvent to get a solution. Lime is dispersed in water and is added to the solution. The solution is kept above 40° C. and stirred till precipitate is formed. The precipitate is removed and dried to obtain a diarylheptanoid complex of calcium with embedded essential oil in the agglomerates. The composition contains curcuminoids complex of calcium but the composition of bisdemethoxycurcumin and demethoxycurcumin is higher than curcumin. Calcium salts could also be used instead of limestone for the complex formation, a person skilled in the art will be capable of using other salts. In one embodiment, the eggshell is used as a source of calcium and other cations.

Another aspect of the invention is that the diarylheptanoid metal complex with embedded lipids/essential oils showed better medical properties than its counterpart, diarylheptanoid. Studies were conducted on animals with diarylheptanoid complexes of Calcium.

A method to improve the general health of poultry birds by administering an effective dose of diarylheptanoid complex of Formula-I. The general health of poultry birds includes but is not limited to reduced mortality, improved weight gain, improvement in meat quality, preventing hypocalcaemia, improvement in bone density, improved eggshell thickness, improved immunomodulatory activity, improved hepatoprotective activity, and improved the analgesic property. In an embodiment, the diarylheptanoid complex showed significant results in treating coccidia infection.

In one embodiment efficacy of diarylheptanoid metal complex as preventatives for Histomonas or blackhead disease in turkeys was studied. Diarylheptanoid metal complex was found to be effective in bringing down the inflammation of the intestines and liver caused due to Histomonasmeleagridis. Histomonasmeleagridis is a protozoan parasite of various galliform birds causing a type of enterohepatitis (inflammation of the intestines and liver) termed histomonosis or ‘blackhead disease’.

In another embodiment efficacy of different doses of diarylheptanoid metal complex on production, immunity and gut health in broiler chicken challenged with Eimeria species was studied. The study also determines the efficacy of diarylheptanoid metal complex on productive, immunological, welfare and gut health attributes of broilers challenged with Eimeria species under cage and deep litter housing systems. Parameters analysed include Immunological determinants, Haematological and serological analysis, Lesion and histological scores and Physical observations.

A method to improve the general health of animals by administering an effective dose of diarylheptanoid complex of Formula-I. The general health of animals includes but is not limited to reduced mortality, improved weight gain, improvement in meat quality, preventing hypocalcaemia, improvement in bone density, improved immunomodulatory activity, preventing udder inflammation in milch animals, treating mastitis, improved hepatoprotective activity, and improved the analgesic property.

In one embodiment efficacy and immunomodulatory ability of diarylheptanoid metal complex against mastitis were studied. The parameters analysed are blood serum calcium, blood serum proteins, complete blood count, Calcium phosphorous ratio, Cytokines, Immunoglobulins, Serum albumin, Vitamin D3, Nutrient absorption, Milk quality (Fat %. SNF%, Lactose, Coliforms Count (E. coli) milk protein casein, whey proteins), Curcumin content in milk, Rumen fluid protozoa etc.

A method to improve the general health of humans by administering an effective dose of diarylheptanoid complex of Formula-I. The general health of humans includes but is not limited to preventing hypocalcaemia, improvement in bone density, improved immunomodulatory activity, preventing drug-induced dyslipidemia, treating stress, treating Alzheimer's disease, antioxidant activity and improving the analgesic property. In an embodiment, the diarylheptanoid complex has better bioavailability than regular diarylheptanoid, further, the oil embedded diarylheptanoid complex has greater bioavailability than just a diarylheptanoid complex.

In one embodiment efficacy of diarylheptanoid metal complex on deworming in cows was studied. Cows positive for Amphistome ova were selected and treated with diarylheptanoid metal complex. Deworming in cows is important for several reasons. First, internal parasites or worms can cause significant harm to cow health and productivity. These parasites can cause damage to the cow's gastrointestinal tract, leading to reduced feed intake, weight loss, and poor growth rates. In severe cases, internal parasites can even lead to death. Second, internal parasites can also have economic impacts on the farm. Infected cows may have reduced milk production and lower-quality meat, which can lead to financial losses for the farmer. Third, internal parasites can also be a source of infection for other animals on the farm. Some parasites can be passed from cow to cow or from cow to other species such as sheep or goats. After 30 days of treatment with diarylheptanoid calcium complex, the number of Ova in the dung matter is nil compared to untreated control.

In another embodiment hepatoprotective effect of diarylheptanoid metal complex is studied in philaleria affected cows. “Hepatoprotective” refers to substances or medications that can help protect the liver from damage and disease. SGOT (serum glutamic-oxaloacetic transaminase) and SGPT (serum glutamic-pyruvic transaminase) are two liver enzymes. Elevated levels of these enzymes can indicate liver damage or disease. After administering diarylheptanoid calcium complex to animals, the SGOT and SGPT levels decreased to normal range.

In another embodiment effect of diarylheptanoid calcium complex on immunity in cows is studied. IgG values in cows after calving refer to the levels of immunoglobulin G (lgG) antibodies in the blood of cows in the period immediately after they have given birth. IgG is a type of antibody that plays a crucial role in the immune system of cows, just as it does in other mammals, including humans. Animals treated with diarylheptanoid calcium complex showed an increase in IgG values.

In another embodiment effect of diarylheptanoid calcium complex on mortality is studied in broiler chickens. Mortality in broiler chickens can occur due to various reasons, including diseases, poor nutrition, environmental stress, and inadequate management practices. Environmental stress factors such as high temperatures, low humidity, poor ventilation, overcrowding, and exposure to harmful gases like ammonia can also cause mortality in broiler chickens. Chickens fed with diarylheptanoid calcium complex, mortality is reduced to 3% as compared to standard drug which showed 5% mortality.

In another embodiment efficacy of diarylheptanoid calcium complex on weight gain in piglets is studied. The average weight of a newborn piglet is approximately 1.1 to 1.5 kilograms. After administration of diarylheptanoid calcium complex to piglets, the average body weight is increased to 2.5 kg on the 15th day and up to 11.1 kg on the 45ₜₕ day.

In another embodiment effect of diarylheptanoid calcium complex for preventing hypocalcemia after calving is studied in cows. Hypocalcemia, also known as milk fever, is a common metabolic disorder in dairy cows. It occurs when the levels of calcium in the blood fall below normal levels, typically around the time of calving. During this time, the demand for calcium increases rapidly, as the cow produces colostrum and milk for her newborn calf. If the cow cannot meet this demand through dietary intake or mobilization of calcium from her bones, hypocalcemia can occur. Supplementation of diarylheptanoid calcium complex showed an increase in serum calcium level compared to untreated control.

In another embodiment effect of diarylheptanoid calcium complex for improving the shell quality is studied in calcium deficient laying hens. Supplementation of diarylheptanoid calcium complex showed an increase in the percentage of normal eggs as compared to untreated control group. Percentage of Cracked eggs, shell less eggs, and corrugated eggs are decreased in diarylheptanoid calcium complex administered group compared to untreated control.

In another embodiment effect of diarylheptanoid calcium complex as a preventive measure to reduce the incidence of udder inflammation is studied in dairy cows. Supplementation of diarylheptanoid calcium complex maintained the White blood cells×10³/μL level in the range of 4-12×10³/μL, which confirms that udder is not infected. The % of Neutrophils is also in the range of 15-33% which shows no damage to tissues in udder.

### EXAMPLES

Specific embodiments of the invention will now be demonstrated by reference to the following general methods of manufacture and examples. It should be understood that these examples are disclosed solely by way of illustrating the invention and should not be taken in any way to limit the scope of the present invention. They are in no way limiting on the preceding description.

### Example 1

### Method of Preparation of Diarylheptanoid Calcium Complexes With Embedded Oil

10 Kg Curcuminoids and 30 Kg Essential oil of turmeric are dissolved in 60 L solvent to obtain the first solution. 1 Kg lime was dispersed in 2 L of water to obtain a lime solution. The lime solution is slowly added to the first solution to obtain a master solution. The master solution is stirred for 2-3 h at room temperature. The precipitate was formed, and the precipitate was filtered and washed with water followed by methanol. The precipitate was dried at 80° C. to form diarylheptanoid calcium complexes. The dried precipitate has the desired composition and it contained at least about 80% diarylheptanoid calcium complexes and at least about 20% oil. The composition obtained in a free-flowing, reddish-brown powder, does not stain the surface

### Example 2

### Method of Preparation of Diarylheptanoid Zinc Complexes With Embedded Oil

10 Kg Curcuminoids and 30 Kg Essential oil of turmeric are dissolved in 60 L solvent to obtain the first solution. 10 Kg Zinc Salt was dispersed in 25 L of water to obtain a solution. The Zn salt solution is slowly added to the first solution followed by 15 Kg sodium bicarbonate dissolved in 100 L water to obtain a master solution. The master solution is stirred for 1-2 h at room temperature. The precipitate was formed, and the precipitate was filtered and washed with water followed by methanol. The precipitate was dried at 80° C. to form diarylheptanoid Zinc complexes. The dried precipitate has the desired composition and it contained at least about 80% diarylheptanoid zinc complexes and at least about 20% oil. The composition obtained in a free-flowing, orange yellow powder, does not stain the surface.

### Example 3

### Method of Preparation of Diarylheptanoid Calcium Complexes With Embedded Oil From Turmeric Rhizome

In a reactor, 100 Kg turmeric rhizome was charged and extracted with 300 L of ethyl acetate repeatedly to obtain a residue and a supernatant. The supernatant was filtered out, and the solvent was stripped from the supernatant to obtain the oleoresin. oleoresin was dissolved in methanol. 1 Kg Lime was dispersed in 2 L water and added slowly to the supernatant solution. The slurry obtained was stirred for 2-3 hrs at room temperature. The precipitate was formed. and the precipitate was filtered and washed with water followed by methanol. The precipitate was dried at 80° C. to form diarylheptanoid calcium complexes. The dried precipitate has the desired composition and it contained at least about 30% diarylheptanoid calcium complexes and at least about 20% oil. The composition obtained in a free-flowing, does not stain the surface.

### Example 4

Method of Preparation of Diarylheptanoid Calcium Complexes With Embedded Oil From Turmeric Rhizome

In a reactor, 100 Kg of turmeric rhizome was charged and extracted with 300 L of ethyl acetate repeatedly to obtain a residue and a supernatant. The supernatant was filtered out, and the solvent was stripped from the supernatant to obtain the oleoresin. The oleoresin was cooled to 4° C. to crystalized the curcuminoids. Curcuminoids were removed from the oleoresin and the remaining oleoresin was dissolved in methanol. 1 Kg Lime was dispersed in 2 L water and added slowly to the methanol solution. The slurry obtained was stirred for 2-3 hrs at room temperature. The precipitate was formed, and the precipitate was filtered and washed with water followed by methanol. The precipitate was dried at 80° C. to form diarylheptanoid calcium complexes. The dried precipitate has the desired composition and it contained at least about 10% diarylheptanoid calcium complexes and at least about 20% oil. The composition obtained in a free-flowing, does not stain the surface.

### Example 5

### Effect of Diarylheptanoid Calcium Complex on Deworming in Cows

Twenty cows that were positive for Amphistome ova were selected for this study. Cows were grouped into four groups with 5 cows in each group. Cows were segregated as follows.

| | |
|---|---|
| Group 1 | Untreated control |
| | Dosage: without oral administration of any drug |
| Group 2 | Positive control (Albendazole) |
| | Dosage: 1.5 g administered for only one day |
| Group 3 | Regular Turmeric extract group |
| | Dosage: 1 g supplement/day for 30 days |
| Group 4 | diarylheptanoid calcium complex |
| | Dosage: 1 g supplement/day for 30 days |

Before and after 30 days of treatment ova was checked from the dung matter.

TABLE 1
Effect of diarylheptanoid calcium complex on deworming in cows.

Ova

| | Before | After |
|---|---|---|
| Group 1 | + + + + + | + + + + |
| Group 2 | + + + + + | − |
| Group 3 | + + + + + | + + |
| Group 4 | + + + + + | − |

Deworming is an important aspect of cow health management that involves the use of medications to control and prevent internal parasites or worms. Internal parasites can negatively impact cow health by causing reduced feed intake, weight loss, and even death in severe cases.

After 30 days of treatment with diarylheptanoid calcium complex, the number of Ova in the dung matter was nil compared to the untreated control. In group 3 animals treated with regular turmeric extract showed some effect for reducing the ova whereas the diarylheptanoid calcium complex administered group showed a complete reduction in the number of ova as compared to the standard drug Albentazole.

### Example 6

### Hepatoprotective Effect of Diarylheptanoid Calcium Complex

The hepatoprotective effect of the diarylheptanoid calcium complex was studied in philaleria-affected cows. 20 cows were selected and divided into four groups. Group 1 is untreated control. Group 2 was fed with the standard drug Liv 52 in a dose of 1.5 g daily for 15 days. Group 3 animals were fed with regular turmeric extract in a dose of 1 g supplement per day for 15 days. Animals in group 4 were fed with diarylheptanoid calcium complex in a dose of 1 g supplement per day for 15 days. After 15 days of treatment, SGOT and SGPT were analyzed.

TABLE 2
Effect of diarylheptanoid calcium
complex on liver condition in cows.

| | | After | | |
|---|---|---|---|---|
| | Before | SGOT | Before | After |
| | SGOT(IU/L) | (IU/L) | SGPT(IU/L) | SGPT(IU/L) |
| Group 1 | 88 | 86 | 255 | 251 |
| Group 2 | 84 | 14 | 262 | 45 |
| Group 3 | 81 | 45 | 278 | 88 |
| Group 4 | 90 | 13 | 254 | 38 |

After administering diarylheptanoid calcium complex to group 4 animals, the SGOT level decreased to the normal range compared to the untreated control group. In group 3 animals treated with regular turmeric extract, the SGOT levels were not decreased to the normal range after 15 days of treatment.

SGPT levels in group 4 animals were reduced to normal levels as compared to standard drug Liv 52.

### Example 7

### Effect of Diarylheptanoid Calcium Complex on Immunity

6 years old pregnant cows were selected and divided into 4 groups with 5 cows in each group. Diarylheptanoid calcium complex and regular turmeric extract (1 g supplement) and standard drug Immunol X (50 ml/cow/day) were fed to cows of each group 4 months prior to calving. IgG values were checked 3 days after calving. Cows are classified as given below.
Group 1: Untreated control
Group 2: Positive control (Immunol X, 50 ml/cow/day)
Group 3: Regular turmeric extract (1 g/day)
Group 4: diarylheptanoid calcium complex (Dosage: 1 g supplement/day)

TABLE 3
Effect of diarylheptanoid calcium complex on immunity in cows

| | IgG (g/L) |
|---|---|
| Group 1 | 38 |
| Group 2 | 73 |
| Group 3 | 60 |
| Group 4 | 76 |

IgG values in cows after calving refer to the levels of immunoglobulin G (IgG) antibodies in the blood of cows in the period immediately after they have given birth. IgG is a type of antibody that plays a crucial role in the immune system of cows, just as it does in other mammals, including humans. Typically, IgG values in cows after calving are measured to assess the quality of the colostrum and the effectiveness of the passive transfer of immunity from the cow to the calf.

Group IV animals fed with diarylheptanoid calcium complex showed an increase in IgG values of 76 g/L. Whereas in untreated control group, the IgG level was 38 g/L.

### Example 8

### Effect of Diarylheptanoid Calcium Complex on Mortality in Birds

4000 broiler chickens under stressed conditions were selected for the study. Chickens were divided into 4 groups with 1000 chickens in each group. Group 1 is untreated control. Group 2 chickens were fed with standard drug Groviplex (0.05 ml/bird/day) for 42 days. Group 3 chickens were fed with regular turmeric extract in a dose of 5 mg/bird/day along with feed per day for 42 days. Chickens in group IV were fed with diarylheptanoid calcium complex in a dose of 5 mg/bird/day along with feed per day for 42 days.

TABLE 4
Effect of diarylheptanoid calcium complex
on mortality in poultry birds.

| | Mortality |
|---|---|
| Group 1 | 25% |
| Group 2 | 5% |
| Group 3 | 10% |
| Group 4 | 3% |

Mortality in broiler chickens can occur due to various reasons, including diseases, poor nutrition, environmental stress, and inadequate management practices. In the untreated control group, after 45 days, the mortality was 25%. In the treatment group fed with diarylheptanoid calcium complex, mortality was reduced to 3% as compared to standard drug which showed 5% mortality.

### Example 9

### Weight Gain Study in Piglets Using Diarylheptanoid Calcium Complex

Eighty piglets were selected for the study. They were divided into four groups of 20 piglets in each group. Group 1 is the normal control group. Group 2 is fed with standard drug Fat plus 1 g/piglet/day along with creep feed. Groups 3 and 4 were fed with regular turmeric extract and diarylheptanoid calcium complex 500 mg/piglet/daily along with creep feed. Weight gain was noted on the 15^{th} day and the 45^{th} day after feeding.

TABLE 5
Effect of diarylheptanoid calcium
complex on weight gain in piglets

Weight gain (Kg)

| | 15^{th} day | 45^{th} day |
|---|---|---|
| Group 1 | 1.8 | 8.5 |
| Group 2 | 2.4 | 10.2 |
| Group 3 | 2.1 | 9.1 |
| Group 4 | 2.5 | 11.1 |

The average weight of a newborn piglet is approximately 1.1 to 1.5 kilograms. After administration of diarylheptanoid calcium complex to piglets, the average body weight was increased to 2.5 kg on the 15^{th} day and up to 11.1 kg on the 45^{th} day. Whereas in the normal control group, the average body weight gain is 1.8 kg on the 15^{th} day and 8.5 kg on the 45^{th} day. The standard drug also gives approximately the same weight gain as the diarylheptanoid calcium complex.

### Example 10

Effect of diarylheptanoid calcium complex for preventing hypocalcaemia after calving. The study was conducted in a dairy farm with 20 cows in third and four parity stage which is divided into 4 groups of 5 cows in each group. Normal calcium level in cow is 8.5 to 10.5 mg/dl. First group was untreated control and second group was cows treated with standard drug mifex 100 ml/cow twice daily. Third group was fed with 1 g regular turmeric extract supplement and fourth group fed with 1 g supplement of diarylheptanoid calcium complex for 30 days.

TABLE 6
Effect of diarylheptanoid calcium
complex on Serum Calcium levels.

| | Serum calcium levels (mg/dl) |
|---|---|
| Group 1 | 1.8 |
| Group 2 | 9.8 |
| Group 3 | 2.1 |
| Group 4 | 10.1 |

Hypocalcemia is a common metabolic disorder that can occur in cows during the first few days after giving birth when the demand for calcium to produce milk exceeds the cow's ability to mobilize calcium from her bones. Supplementation of diarylheptanoid calcium complex showed an increase in serum calcium level compared to untreated control.

### Example 11

### Evaluation of the Effect of Diarylheptanoid Calcium Complex on Improving the Eggshell Quality in Layers

4000 laying hens between the ages of 50 and 60 weeks that were deficient in calcium were chosen for the study. Hens were divided into 4 groups with 1000 hens in each group. Group 1is untreated control. Group 2 hens were fed with the standard drug Himcal (0.05 ml/bird/day) for 45 days. Group 3 hens were fed with regular turmeric extract in a dose of 100ppm/bird/day along with feed per day for 45 days. Hens in group IV were fed with diarylheptanoid calcium complex in a dose of 100 ppm/bird/day along with feed per day for 45 days. Shell quality was analysed by Candling method.

TABLE 7
Effect of diarylheptanoid calcium complex on Shell quality.

| | | Normal | Cracked | Shell less | corrugated |
|---|---|---|---|---|---|
| | | eggs(%) | Egg(%) | egg (%) | eggs(%) |
| Group | Before | 30% | 20% | 30% | 20% |
| 1 | After | 30% | 30% | 25% | 15% |
| Group | Before | 35% | 25% | 25% | 15% |
| 2 | After | 74% | 10% | 8% | 8% |
| Group | Before | 30% | 20% | 30% | 20% |
| 3 | After | 35% | 20% | 30% | 15% |
| Group | Before | 35% | 25% | 25% | 15% |
| 4 | After | 80% | 9% | 6% | 5% |

The quality of the eggshell is paramount to both producers and consumers. In particular, it is very important in upholding the hygiene of eggs, as any damages or cracks will make the eggs more susceptible to bacterial contamination. In group 4 hens, after administering diarylheptanoid calcium complex for 45 days, the percentage of normal eggs improved by 80% as compared to untreated control for which the normal egg is only 30%. Cracked eggs, shell less eggs, and corrugated eggs percentages were also decreased compared to untreated control.

### Example 12

### Effect of Diarylheptanoid Calcium Complex Used as a Preventive Measure to Reduce the Incidence of Udder Inflammation in Dairy Cows

The study was conducted in a dairy farm with 20 Milch & Lactating Cows which is divided into 4 groups of 5 cows in each group. Normal White blood cells, WBC in cows are 4×10³/μL to 12×10³/μL and normal % Neutrophils is around 15-33%.

First group was untreated control and second group was cows treated with standard supplement MINTREX® chelated trace minerals at 1 gram per day per head dosage. Third group was fed with 1 g regular turmeric extract supplement and fourth group fed with 1 g supplement of diarylheptanoid calcium complex for 30 days.

TABLE 8
Effect of diarylheptanoid calcium complex on White
blood cells × 10³/μL levels.

| | White blood cells × 10³/μL |
|---|---|
| Group 1 | 36.1 × 10³/μL to 40 × 10³/μL |
| Group 2 | 15 × 10³/μL to 22 × 10³/μL |
| Group 3 | 17 × 10³/μL to 22 × 10³/μL |
| Group 4 | 5 × 10³/μL to 11.1 × 10³/μL |

TABLE 9
Effect of diarylheptanoid calcium complex on Neutrophils levels.

| | White blood cells × 10³/μL |
|---|---|
| Group 1 | 50-60% |
| Group 2 | 39-49% |
| Group 3 | 40-45% |
| Group 4 | 15-30% |

When the udder is infected, white blood cells move to the udder and into the milk to defend against the invading bacteria. This process is very important; without it, the elimination of even mild cases of udder inflammation would be very slow, with tissue damage increased. Supplementation of diarylheptanoid calcium complex maintained the White blood cells in the range of 4-12×10³/μL, which confirms that the udder was not infected. The percentage of Neutrophils was also in the range of 15-33% which shows no damage to tissues in the udder.

We have bought out the novel features of the invention by explaining some of its preferred embodiments thereby enabling any person skilled in the art to understand and visualize our invention. It is also to be understood that the above invention is not limited in its application to the details set forth in the above description or illustrated in the drawings. The phraseology and terminology employed herein are for the purpose of description only and should not be regarded as limiting. Although the invention has been preferred embodiments thereof, variations and modifications can be affected within the scope of the invention as described herein above and as defined in the appended claims.

1. A medicinal preparation comprising a free-flowing stain free composition having a mixture of diarylheptanoid complex and essential oil of turmeric embedded in diarylheptanoid metal complex wherein the diarylheptanoid complex is having the Formula. 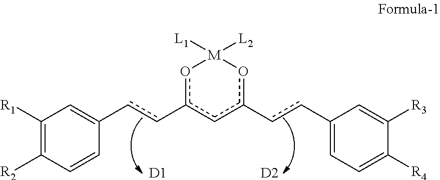
Wherein, R₁, R₂, R₃, and R₄ are selected from a group consisting of: hydrogen, —OH, —OCH₃, —OCOCH₃, —OCO—CHR—NH₂, O-Piperoyl, —O-galloyl and —O-terpenoid.
L1 and L2 are selected from a group consisted of —OH, —OH₂, CI⁻, amine, Diarylheptanoids, flavonoids, phenolic acids, stilbenes, saponins and tannins;
D1 and D2 are selected from a group consisting of single and double bond; and
M is selected from a group consisting of alkali metals, alkaline earth metal, transition metals, lanthanides, and actinides; and
optionally further comprising a pharmaceutically acceptable carrier or diluent.

2. The medicinal preparation comprising a free-flowing stain free composition as claimed in claim 1, wherein said composition is having at least 5% oil of turmeric by weight.

3. The medicinal preparation comprising a free-flowing stain free composition as claimed in claim 1, wherein said composition is having 5 to 40% essential oil turmeric by weight embedded in the diarylheptanoid complex.

4. (canceled)

5. The medicinal preparation comprising a free-flowing stain free composition as claimed in claim 1, wherein curcuminoids complex makes 1% to 90% of said composition.

6. The medicinal preparation comprising a free-flowing stain free composition as claimed in claim 1, wherein curcuminoids complex makes up 60% to 90% of said composition.

7. (canceled)

8. The medicinal preparation comprising a free-flowing stain free composition as claimed in claim 1, wherein the medicinal preparation comprises a pharmaceutically acceptable carrier or diluent, and is administrable orally, parenterally or topically to avians and mammals.

9. The medicinal preparation comprising a free-flowing stain free composition as claimed in claim 8, wherein avians are ostriches, chickens, turkey, quail, ducks, flamingos, grebes, pigeons.

10. The medicinal preparation comprising a free-flowing stain free composition as claimed in claim 8, wherein mammals are rats, cats, dogs, bats, sheep, horse, camel, goat, cows, pigs and humans.

11. The medicinal preparation comprising a free-flowing stain free composition as claimed in claim 1 wherein the medicinal preparation comprises a pharmaceutically acceptable carrier or diluent, and is administrable orally in the form of tablets, capsules, granules, powders, lozenges, syrups, elixirs, solutions, and suspensions following the standard pharmaceutical practice.

12. The medicinal preparation comprising a free-flowing stain free composition as claimed in claim 1, wherein the carriers is selected from gelatine, lactose, sodium citrate, salts of phosphoric acid, starch, magnesium stearate, sodium lauryl sulphate, talc, and polyethene glycol.

13. The medicinal preparation comprising a free-flowing stain free composition as claimed in claim 1, further comprising one or more of binders, buffer agents, preservatives, sweetening agents, flavouring agents, glazes, disintegrators, coating agents, emulsifying agents, and suspending agents.

14. The medicinal preparation comprising a free-flowing stain free composition as claimed in claim 8, wherein parenteral administration is selected from intramuscular, intraperitoneal, subcutaneous and intravenous.

15. The medicinal preparation comprising a free-flowing stain free composition as claimed in claim 1, wherein the effective dosage for an adult will be between 50-1000 mg two times a day.

16. A method of preparation of a free-flowing stain free composition having a mixture of diarylheptanoid complex and essential oil of turmeric embedded in diarylheptanoid metal complex wherein the diarylheptanoid complex has the structure of Formula-1, comprising of the steps:
a) extracting the cleaned Turmeric rhizome with an organic solvent, preferably with ethyl acetate to obtain a solvent part and residue part;
b) separating the solvent part from the residue, and concentrate to obtain the oleoresin;
c) cooling said oleoresin to a temperature of 4° C. to crystalize curcuminoids separating the curcuminoid crystals from the oleoresin;
d) dissolving the remaining oleoresin in a methanol to get a solution;
e) adding a source of calcium dispersed in water to the resultant solution of step d);
f) maintaining the resultant solution at a temperature of 27° C. and stirring till precipitate is formed; and
g) separating the precipitate, washed with water and methanol and drying to obtain a diarylheptanoid complex of calcium with embedded essential oil in the agglomerates.

17. The method of preparation of a free-flowing stain free composition as claimed in claim 16, wherein source of Calcium is selected from lime, calcium salts and calcium oxide made from egg shell.

18. The method of preparation of a free-flowing stain free composition as claimed in claim 16, wherein the organic solvent used in the extraction of Turmeric rhizome is ethyl acetate.

19. The method of preparation of a free-flowing stain free composition as claimed in claim 16, wherein the solvent used for dissolving the remaining oleoresin in step d) is methanol.

20. The medicinal preparation comprising a free-flowing stain free composition as claimed in claim 1, wherein, upon administration to an avian or mammal, the medicinal preparation controls worms, improves the liver conditions, improves the immunity, reduces the mortality, improves weight gain, or prevents hypocalcaemia.

21.-25. (canceled)

26. The medicinal preparation comprising a free-flowing stain free composition as claimed in claim 1, wherein, upon administration to an avian, the medicinal preparation improves egg shell quality.

27. The medicinal preparation comprising a free-flowing stain free composition as claimed in claim 1, wherein, upon administration to a mammal, the medicinal preparation prevents the incidence of udder inflammation.
